(19) United States
(12) Patent Application Publication
(10) Pub. No.: US 2005/0033384 A1
(43) Pub. Date: Feb. 10, 2005

(54) Cochlear ear implant

(76) Inventor: Mike K. Sacha, Chanhassen, MN (US)

Correspondence Address: DRUMMOND & DUCKWORTH, Suite 440, East Tower, 5000 Birch Street, Newport Beach, CA, 92660, US

(21) Appl. No.: 10/911,185

(22) Filed: Aug. 4, 2004

Related U.S. Application Data
(60) Provisional application No. 60/492,347, filed on Aug. 4, 2003.

(51) Int. Cl. A61N 1/18

(52) U.S. Cl. 607/57

(57) ABSTRACT

A simple cochlear implant is provided that can be implanted in a doctor's office under local anesthesia, which does not destroy residual hearing, and in a preferred embodiment, which is small enough to fit within a person's ear canal. The cochlear ear implant includes an exterior ear module, an interior ear module, and a cochlear electrode array. The exterior ear module includes a hollow housing within which are located the active electrical components including a microphone, power supply, and processor. The exterior ear module is easily removable from the body without surgery and is positioned in the auditory canal, the concha bowl or behind the pinna. The interior ear module is a semi-permanent assembly located immediately exterior to the tympanic membrane. It is a simple passive module for relaying signals to the electrode array. The communication of auditory signals from the exterior ear module to the interior ear module may be achieved by various techniques including by direct electrical transmission. However, the communication between the exterior and interior ear modules is preferably accomplished using a transcanal induction link. The electrode array extends from the interior ear module through the tympanic membrane to engage the cochlea. The electrode array includes an implanted active electrode, a return electrode, and a biocompatible miniature connector for connecting to the interior ear module.

### RELATED APPLICATIONS

This application is a continuation-in-part of my co-pending U.S. Provisional Application Ser. No. 60/492,347, filed Aug. 4, 2003.

### BACKGROUND OF THE INVENTION

The present invention relates to a transcanal, transtympanic cochlear implant device ideally suited for those profoundly deaf, where conventional hearing aids are of limited or no value. A profoundly deaf ear is typically one in which the sensory receptors of the inner ear, called hair cells, are damaged or diminished. Unfortunately, the use of a hearing aid does not enable such an ear to process sound. Meanwhile, cochlear implants bypass damaged hair cells and directly stimulate the hearing nerves with an electrical current, allowing individuals who are profoundly or totally deaf to hear.

The ear is an amazing structure consisting of three main parts including the outer ear, the middle ear and the inner ear. The outer ear includes the visible outer portion of the ear called the auricle and the auditory canal. The middle ear includes the eardrum and three tiny bones commonly referred to as the “hammer”, “anvil” and “stirrup”, and medically referred to as the “malleus”, “incus” and “stapes”. The inner ear comprises the fluid filled coil-shaped cochlea which contains thousands of tiny hair cells.

When the ear is functioning normally, sound waves are collected by the outer ear and directed through the ear canal to the middle ear. The sound waves strike the eardrum, also called the tympanic membrane, and cause it to vibrate. This vibration creates a chain reaction in the three tiny bones of the middle ear. Motion of these bones causes movement of the fluid within the cochlea. Meanwhile, the hair cells within the cochlea convert these mechanical vibrations into electrical impulses which are sent to the hearing nerves. Thereafter, the hearing nerves transmit electrical energy to the brain which interprets the energy as “sound”.

Unfortunately, some people suffer damage or depletion of the hair cells resulting in profound hearing loss. In these cases, electrical energy cannot be generated and transmitted to the brain. Without these electrical impulses, the hearing nerves cannot carry messages from the cochlea to the brain and even the loudest of sounds cannot be heard.

Cochlear implants have been developed to enable those persons suffering from profound hearing loss to hear. Although the hair cells in the cochlea may be damaged, there are usually some surviving hearing nerves. A cochlear implant works by bypassing the damaged hair cells and directly stimulating the surviving hearing nerves with an electrical signal. The stimulated hearing nerves then carry the electrical signals to the brain which are interpreted by the brain as sound.

Typically, cochlear implants include two modular units. The first unit is an external module which typically resides behind the ear auricle, in the temporal bone region. It includes external microphones that sense acoustic pressure waves and then converts them to electrical signals. The electrical signals are processed by a signal processor, which typically includes amplification and conversion, into stimulation signals. The second module is an implanted unit which is located in a temporal bone excavation typically located just behind the auricle. The outer module communicates with the implanted module primarily via transcutaneous induction. Across this inductive link, audio information is transmitted as well as energy to power the electronics of the implanted module. Within this implanted module, algorithms are implemented that allow for various methodologies of electrode stimulation. The implanted module includes an electrode array which extends from the excavated area to the cochlea, where the array end is implanted within the scala tympani duct. This nerve stimulation is then interpreted by the brain as sound.

Unfortunately, cochlear implants suffer from significant drawbacks. The main problem with conventional cochlear implants is that during the implantation phase, residual hearing can be destroyed. Since the length of typical electrode arrays extend beyond the first cochlear bend, it is forced into the curvature by deflecting off the cochlear wall, causing damage to the Stria Vascularus, Spiral Ligament, and even the Basilar Membrane regions. This damage, potentially, precludes these patients from utilizing future technological developments in hearing science.

Another problem is that traditional cochlear implants require temporal bone excavation, within which the implanted electronics module is placed and through which the electrode array is presented-to the cochlea. To accomplish this, the cochlear implants must be surgically introduced via a complicated and risky procedure known as the facial recess mastoidectomy. This operation requires the patient to be placed under general anesthesia which represents an additional risk. In addition, patients that cannot tolerate general anesthesia are excluded from participating in this technological development.

Another problem with traditional cochlear implants is their complexity. Most conventional systems require active electronics in both the external module and the implanted module. This requires the inductive link to transfer power as well as audio information, simultaneously, to the implanted module, thus increasing the complexity of the overall system.

Cochlear implants are also very expensive, requiring surgery, anesthesia, hospital stay, and cochlear programming as each cochlear implant must also be programmed individually for each user which is also expensive and time consuming. The entire procedure is prohibitively expensive and impractical for the vast majority of deaf people in the world. Moreover, few doctors in developing countries have the sophistication, expertise and equipment to perform a facial recess mastoidectomy.

Thus, there is a significant need for a cochlear implant which is inexpensive and involves a minimum of invasive surgery. Accordingly, it would be desirable for a simple cochlear implant that can be implanted in a doctor's office, under local anesthesia, that does not destroy residual hearing, and is small enough to fit within a person's ear canal.

### SUMMARY OF THE INVENTION

The present invention addresses the aforementioned disadvantages by providing an improved cochlear implant that does not require significant surgery under general anesthesia, that does not destroy residual hearing, and is small enough to fit within a person's ear canal. The cochlear implant of the present invention includes an exterior ear module, an interior ear module, and an implanted electrode array. The exterior ear module is preferably located in the exterior region of the ear canal, though in alternative embodiments, the exterior ear module may also be located behind the ear. The interior ear module is located in the ear canal immediately exterior to the tympanic membrane. Finally, the wire electrode extends from the interior ear module through the tympanic membrane so that the electrode distal extremity engages the cochlear nerves. The most exterior module, referred to as the exterior ear module, is removable, and communicates audio signals to the semi-permanent interior ear module, which in turn, transmits electrical stimulus signals to the electrode array.

The exterior ear module is a removable hollow shell structure which is preferably located in the exterior region of the auditory canal. The module itself is constructed in the same manner that a conventional In-The-Ear (ITE) hearing aid is constructed. An impression, or casting, is made of a patient's ear canal, followed by a mold fabricated from this casting, and finally a thin casting, or shell, is cast from this mold. The result is an exact shell replica of the patient's ear canal. Within this shell reside the various electrical components including a microphone, an audio processor, and a power source. Because the general location of this shell is within the concha bowl region, the microphone(s) located on the outermost surface can utilize the ear's natural sound gathering properties to help provide valuable spacial cues. Ventilation for the inner ear may also be provided through the exterior ear module by providing longitudinally extending vents.

As opposed to the easily removable exterior ear module, the interior ear module is a semi-permanent assembly located immediately exterior to the tympanic membrane. It is a simple passive module for relaying signals to the electrode array which passes through the tympanic membrane. The interior ear module is of simple construction and does not contain active electronics. Instead, the purpose of the interior ear module is to receive signals from the exterior ear module, which may be pulsatile or amplitude modulated in nature, and convey that signal to the cochlear electrode array. To transmit the signals to the electrode array, the interior ear module also includes a miniature biocompatible connector to which the cochlear electrode makes its connection. The connector should be biocompatible and miniature in construction and provide relative ease of connectability and accessibility for a surgeon.

The housing component of the interior ear module is made from a low to moderate durometer silicone, or other similarly performing material, to help accommodate the variations in ear canal dimensions. Many different size housings may be necessary to fit the range of patients from children to large adults. The unique shape of the housing also allows for aeration of the very exterior portion of the ear canal. Possible structural variations entail manipulating the basic housing shape to accommodate more extreme ear canal structural variations. Moreover, the interior ear module may include ventilation ducts to aerate the very exterior section of ear canal and provide space for the cochlear electrode array connection to attach to the housing mating connector.

The transmission of electrical signals from the exterior ear module to the interior ear module may be achieved by various techniques including direct electrical contact. However, the communication between the exterior and interior ear modules is preferably accomplished using a transcanal induction link. To this end, the exterior ear module includes a primary induction coil that produces a variable electromagnetic field in response to electrical signals sent from the processor which is, in turn, transmitted to the secondary coil through induction. In a preferred embodiment, the primary induction coil is located at the distal end of the exterior ear module's housing for close proximity to the interior ear module. The primary coil is also preferably an air wound solenoid shaped coil wound around a thin plastic tube to create a central cavity.

Meanwhile, the interior ear module includes a secondary coil for the inductive link. The interior ear module also preferably includes a small axially aligned ferrite rod around which the secondary coil is wound. This coil assembly is placed within a custom preformed housing made of silicone, or other similar material. The secondary coil is wound around approximately three quarters of the ferrite core rod's length with the remaining quarter or so protruding from the anterior housing face of the interior ear module. The primary coil, as well its outer shell, has a cavity through which the interior ear module's ferrite core is inserted. The ferrite core rod is sized and protrudes from the interior ear module so as to project into the opening of the exterior ear module, around which the primary core is wrapped.

The inductance of the secondary coil, which can be made relatively large by the utilization of the centrally aligned small ferrite rod, integrates this induction signal and passively extracts audio information transmitted by the primary coil to produce stimulus signals. This method of coupling removes any angular coil coupling issues and greatly diminishes the coupling losses due to coil separation. The inductive coil construction also results in a very efficient energy transfer from outer module to inner module. The efficiency of this inductive coupling is strongly influenced by the proximity of the two coils. The desirable placement of these coils would then be an axial alignment of the two coils placed as close to each other as possible. The unique ability of this design to incorporate a ferrite rod further increases coil coupling by controlling the magnetic lines of flux.

Finally, the third component of the cochlear implant is the electrode array which extends through the tympanic membrane. The electrode array is a simple structure including the implanted active electrode, the return electrode, and a biocompatible miniature connector. A physical connection via the biocompatible miniature connector, connects the electrode to the interior ear module. A specially manufactured connector is necessary due to the physically small size required and the need for biocompatibility. The wire conductor is preferably composed of silver wire. The distal extremity of the implanted electrode is typically inserted into the scala tympany of the cochlea. The return electrode is typically located extracochlearly in the middle ear cavity. This placement of the active and return electrodes is intended to facilitate current spreading and the resultant stimulation of a larger population of neurons. The return electrode may be constructed as a small conductive mesh region to enable a low impedance tissue connection. The preferred wire material for the active and return electrode mesh is also silver. Possible structural variations of the cochlear electrode include manipulating the active electrode shape and orientation to better project the stimulating current towards the modiolus region. Preferably, the physical length of the electrode is approximately 6 mm, much shorter than traditional multielectrode arrays. The shortness of this electrode significantly reduces trauma to the cochlea, minimizing the chances of compromising a patient's residual hearing. The patient is then able to pursue future hearing technological developments.

In operation, the microphone(s) of the cochlear implant convert(s) the ambient sound environment into an electrical analogy. A state of the art digital signal processor (DSP) based audio processor creates the stimulus signals through necessary signal processing and conditioning using amplification, compression, expansion, threshold adjustments and noise canceling algorithms. Most modern DSP processors utilize either pulse position or pulse width modulation methods of outputting a signal, which typically would be routed to the speaker for conversion to acoustic energy. Instead, the output of the audio processor is a pulsatile waveform that is modulated with audio information to power the primary coil of the induction coupling system. The interior ear module, which contains the secondary coil, then integrates this waveform and extracts the embedded audio information. The audio information is then transmitted through the interior ear module to the cochlear electrode array to stimulate the hearing nerves.

To power this cochlear implant, a conventional hearing aid battery can be used, or a rechargeable lithium based, or other chemistry battery. The recharging of this battery can be accomplished using induction methods in which the primary coil, located in the exterior ear module, is configured to receive the induction recharge energy. This feature greatly adds to the user's convenience and ease of operation.

Various modifications of the cochlear implant may be made. For example, the implant may include a means to assist with the removal of the outer module from the ear canal, such as a withdrawal line. A possible functional variation involves the usage of two microphones, instead of one, that provides a cardioid like spacial response pattern to the user. Any improvement in signal-to-noise (S/N) ratio, that results from using directivity, is of major importance to the hearing impaired. By using two microphones configured to provide a cardioid response, the desirable increase in S/N ratio is achieved through spacial filtering. An extension of this would be to provide a pair of microphones, per ear, that would communicate with each other via a low power, miniature RF or inductive link, thus creating a four microphone array. The directionality of such an array would provide for even greater spacial filtering resulting in a further increase in S/N that is so critical for the hearing impaired. A variation of this approach would be to provide for a radio receiver within the exterior ear module that would receive a signal transmitted by a desk-top or handheld directional microphone array. This type of array would provide additional directionality improvement and provide further capability in attenuating unwanted environmental noises.

For patients that cannot accept an in the ear object as large as the exterior ear module, a behind the ear location (BTE), or other around the ear location, can be used. These constructions relocate the relatively bulky exterior ear module, that contains the electronics and other components, outside the ear canal. Preferably, the exterior ear module is moved from the canal region and placed above the Pinna similarly to how a conventional behind the ear (BTE) acoustic hearing aid is worn. From this BTE module, a wire is routed to an exterior ear module where the primary induction coil is located in a new housing-like structure. This new structure couples inductively to an interior ear module, such as discussed above, which contains the secondary coil and connector to which the cochlear array is connected. The interior ear module (housing) is relatively small, affording greater freedom of placement within the canal region. A wire connection from the BTE module (which contains the electronics, battery and other components) is run to a new smaller exterior ear module which only contains the primary coil. This new BTE housing module is placed in proximity to the interior ear module and the secondary coil it contains.

A primary object of the present invention is to provide a simple in-the-ear cochlear implant that will overcome the shortcomings of the prior art devices.

Another object is to provide a simple in-the-ear cochlear implant that is located within the concha bowl and external meatus of the ear where a conventional acoustic In-The-Ear (ITE) hearing aid device is worn.

Another object is to provide a simple in-the-ear cochlear implant that utilizes a simple single contact electrode with which to stimulate the remaining basilar membrane dendrites or spiral ganglia nerve cells.

Another object is to provide a simple in-the-ear cochlear implant that enables much higher coil coupling between the exterior ear module and the interior ear module producing more efficient energy transfer from the external processor unit to the internal module.

Another object is to provide a simple in-the-ear cochlear implant procedure that can be performed entirely through the ear canal in a doctor's office under local anesthesia.

Another object is to provide a simple in-the-ear cochlear implant that provides lower costs and less trauma to the patient.

These and other specific objects and advantages of the invention will be apparent to those skilled in the art from a review of the following detailed description taken in conjunction with the drawings.

### BRIEF DESCRIPTION OF THE DRAWINGS

FIG. 1 is a cutaway view of the human ear with the cochlear implant of the present invention;

FIG. 2 is a second view of the human ear illustrating a second embodiment of the cochlear implant of the present invention residing predominantly in the concha bowl;

FIG. 3 is a sectional view of the human ear illustrating a third embodiment of the cochlear ear implant of the present invention including a behind-the-ear module;

FIG. 4 is a side view illustrating the cochlear ear implant including a behind-the-ear module;

FIG. 5 is an additional side view illustrating the cochlear ear implant of the present invention including a behind-the-ear module;

FIG. 6 is a block diagram illustrating the operation of a cochlear ear implant of the present invention including an amplitude modulator and amplitude demodulator;

FIG. 7 is a schematic view of the ear illustrating a cochlear ear implant of the present invention including an exterior ear module in the concha bowl and including electrical terminals;

FIG. 8 is a side view of the cochlear ear implant including a behind-the-ear module and acoustic speaker;

FIG. 9 is an additional side view of a cochlear ear implant including behind-the-ear module and speaker assembly;

FIG. 10 is an additional side view of a cochlear ear implant including exterior ear module located within the concha bowl and including a speaker;

FIG. 11 provides side and perspective views illustrating various constructions for inductive coupling between the exterior and interior ear modules;

FIG. 12 illustrates a first inductive coil coupling system for use with the cochlear ear implant of the present invention;

FIG. 13 illustrates a second inductive coil coupling system for use with the cochlear ear implant of the present invention;

FIG. 14 illustrates a third inductive coil coupling system for use with the cochlear ear implant of the present invention;

FIG. 15 illustrates a fourth inductive coil coupling system for use with the cochlear ear implant of the present invention;

FIG. 16 is a side view illustrating an electrode array for use with the cochlear ear implant of the present invention; and

FIG. 17 is a block diagram illustrating the various components of the cochlear ear implant of the present invention.

### DETAILED DESCRIPTION OF THE INVENTION

While the present invention is susceptible to the embodiment in various forms, as shown in the drawings, hereinafter will be described the presently preferred embodiments of the invention with the understanding that the present disclosure is to be considered as a exemplification of the invention and is not intended to limit the invention to the specific embodiments illustrated.

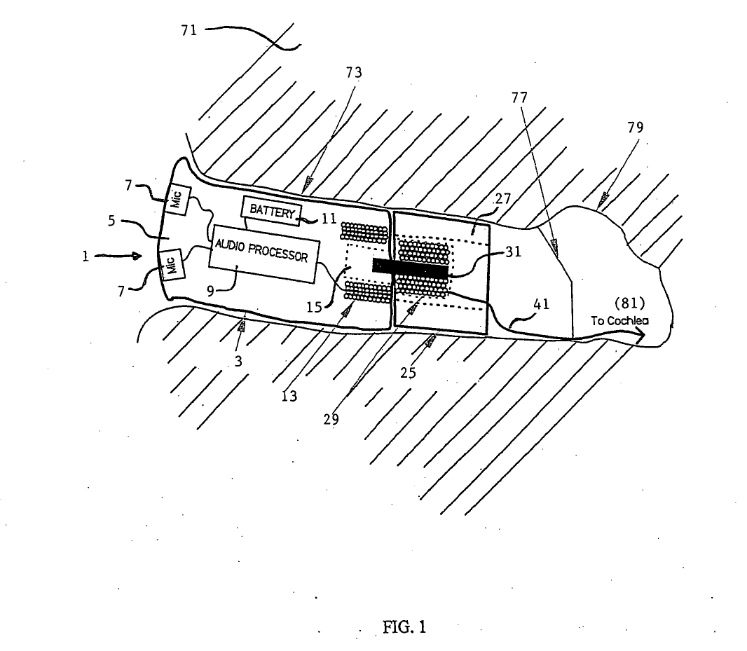

With reference to the figures, the cochlear ear implant 1 of the present invention includes three primary components, namely an exterior ear module 3, an interior ear module 25 and an electrode assembly 41. The exterior module may be constructed in various forms depending on the construction of the patient's ear. For example, for the preferred embodiment of the cochlear ear implant shown in FIG. 1, the exterior ear module 3 is located in the exterior region of the auditory canal 73. The exterior ear module is made in the same manner as a conventional in-the-ear hearing aid. First, an impression or casting is made of the patient's ear canal, followed by typical molding techniques. The result is a housing 5 which presents an exact replica of the patient's ear canal. Located within the exterior ear module's housing 5 are the active components of the cochlear implant. Namely, the exterior ear module 3 houses one or more microphones 7, an audio processor 9, and a power supply 11. In operation, the exterior ear module performs all steps in receiving and conditioning sound waves for transmission to the cochlear electrode array. The conditioning steps may include amplification, filtering, and conversion into stimulus signals which are interpreted by the brain as sound. As will be explained in greater detail below, the stimulus signals may be altered through transmission of the various components, such as the interior ear module. However, the predominant signal processing is preferably conducted within the exterior ear module. The processing may utilize either pulse position or pulse width modulations to stimulate the hearing nerve of the cochlea.

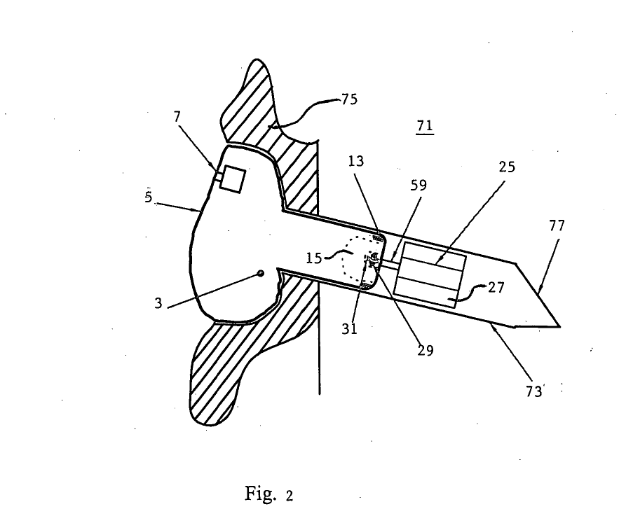

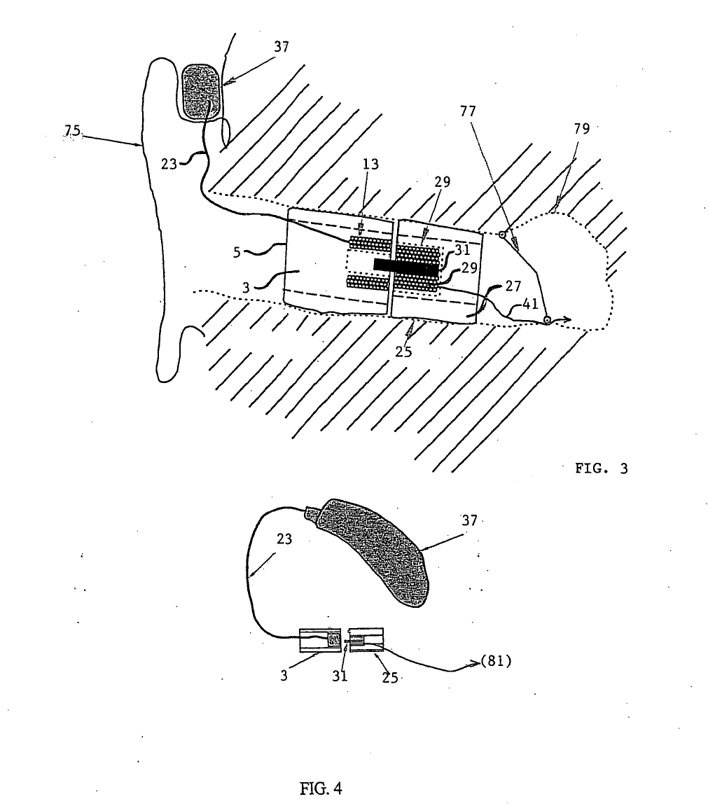

As shown in FIGS. 2 and 3, the exterior ear module 3 may also be constructed to reside within the concha bowl, and to incorporate a behind-the-ear module 37. With reference to FIG. 2, an exterior ear module 3 is sized to reside within the concha bowl of a patient's ear. Again, the exterior ear module's housing 5 is constructed in a similar manner as a hearing aid. Impressions are made of the patient's concha bowl, followed by casting and fabrication of the exterior ear module housing. Again, the microphone, processor, and power supply are located within the exterior ear module 3. Advantageously, the concha bowl in-the-ear construction provides additional space and corresponding savings and increases in quality in component performance compared to the exterior ear module construction located within the auditory canal.

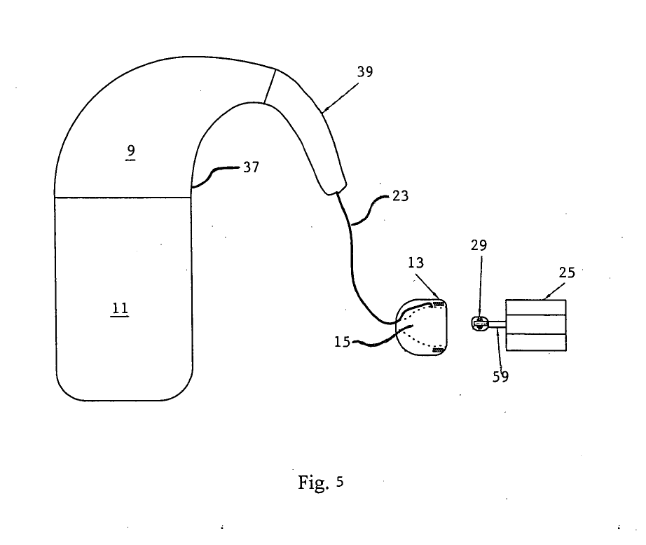

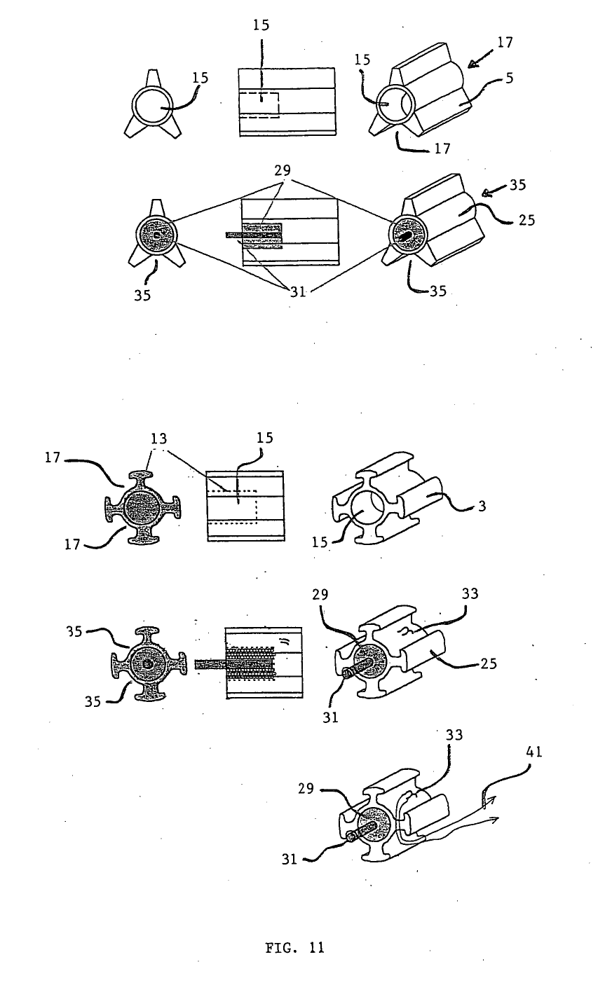

In still an additional embodiment of the present invention, as shown in FIGS. 3-5, the exterior ear module 3 may incorporate an additional behind-the-ear module 37. This construction is considered preferable where patients have extremely small auditory canals, or where a patient is not particularly concerned with the aesthetics of a behind-the-ear module which is more visible to the public. For this embodiment, the microphone, processor and power supply are preferably located within the behind-the-ear module 37. The cochlear implant includes a wire 23 for transmitting auditory information in the form of stimulus signals to the exterior ear module 3. As would be understood by those skilled in the art, the exterior ear module 3 and behind-the-ear module 37 may be constructed in various forms. For example, as shown in FIG. 5, preferably the behind-the-ear module 37 includes an ear hook arm 39 for assisting the module in residing upon a patient's pinna. Moreover, as shown in FIG. 11, the exterior ear module 3 may, or may not, include ventilation vents 17 which extend along the entire length of the module to allow ventilation throughout the patient's auditory canal.

With reference again to all of the figures, the second primary component of the cochlear ear implant 1 of the present invention is a passive interior ear module 25. As opposed to the exterior ear module 3 which is intended to be easily removable from the auditory canal, the interior ear module 25 is intended to be “semi-permanent”. Easily removable is defined herein to include easily removed using finger manipulation or manipulation using surgical tools without significant pain or damage to a person's ear without the use of a local or general anesthesia. Meanwhile, the term “semi-permanent” is intended to mean that the interior ear module can be removed under a local anesthesia or under a minor surgical procedure. Like the exterior ear module 3, the interior ear module 25 may be constructed by making an impression or casting of the interior of the patient's ear canal, substantially adjacent to the tympanic membrane 77. The casting results in the production of a housing 27 having dimensions so as to engage the interior sidewalls of the auditory canal 73.

Preferably, the interior ear module does not contain any active electronics. Instead, its sole purpose is to receive stimulus signals from the exterior ear module 3 which may be pulsatile or amplitude modulated in nature, and convey those signals to the cochlear electrode array 41. To connect to the electrode array 41, the interior ear module 25 includes a biocompatible connector 33. With reference to FIG. 11, as with the exterior ear module 3, the interior ear module 25 may, or may not, include air ventilation shafts 35 for aerating the ear canal 73.

A primary aspect of the invention is that the exterior ear module 3 is selectively connectable and disconnectable to the interior ear module 25 for selectively transmitting auditory signals from the exterior ear module to the interior ear module. The selective electrical coupling between modules can be accomplished by various means. For example, the modules may be electrically connected using wires and simple quick connect/disconnect connectors. Alternatively, as shown in FIG. 7, an electrical connection may be accomplished using a simple mechanical engagement between electrical terminals 63. However, it is believed that the preferred manner for communicating auditory signals from the exterior ear module to the interior ear module is accomplished by an electromagnetic induction link which does not require physical contact between components. As shown in the figures, the exterior ear module 3 is constructed to include a circular primary coil 13 concentrically aligned with the module's central axis. Furthermore, the module includes a central cavity 15.

Meanwhile, the interior ear module 25 includes a secondary coil 29 that produces a variable electric current in response to the variable electromagnetic field produced by electrical signals sent to the primary coil 13. Preferably, the interior ear module 25 also includes a ferrite rod 31 which projects axially outward to project into the exterior ear module's central cavity 15. The use of the ferrite rod increases the efficiency of the inductive coupling and also aids in axial alignment between ear modules 3 and 25.

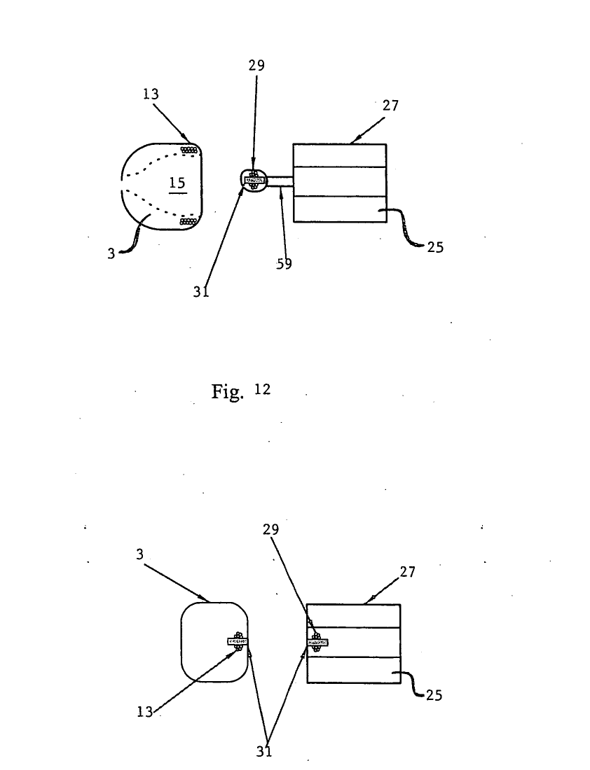

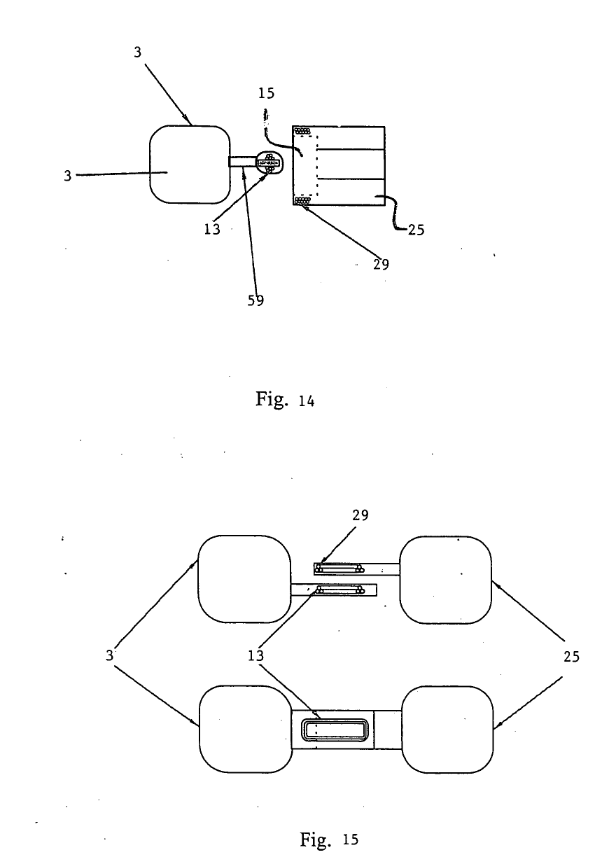

As shown in the figures, the inductive coupling may also take various alternative forms without departing from the spirit and scope of the invention. For example, as shown in FIGS. 5, 9, 10, and 12 a secondary coil may be affixed to the end of a flexible shaft 59. The flexibility of the shaft assists in the alignment of the secondary coil within the primary cavity 15 to provide concentric alignment with the primary coil 15. As shown in FIGS. 13-15, still additional inductive coupling constructions are possible. For example, as shown in FIG. 13, the inductive coupling may include a primary coil and ferrite rod positioned within the exterior ear module 3, while the interior ear module 25 includes a similarly constructed coil assembly including a secondary coil 29 and ferrite rod 31. Alternatively, though most of the above-described constructions include a secondary coil 29 which resides within a cavity and ring construction of the primary coil 13, the inductive coupling may be constructed in a reverse manner in which the primary coil 13 of the removable exterior ear module 3 is sized and positioned to project into a cavity formed within the semi-permanent interior ear module 25. Again, the interior coil, in this case the primary coil, may be positioned at the distal extremity of a flexible shaft 59. In still an additional construction shown in FIG. 15, the individual ear modules 3 and 25 may include laterally extending arms including horizontally aligned coils. Still additional inductive coil constructions can be devised by those skilled in the art.

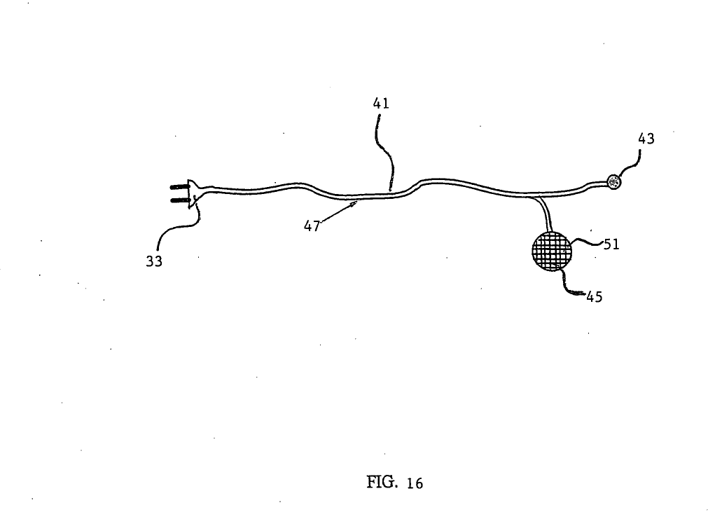

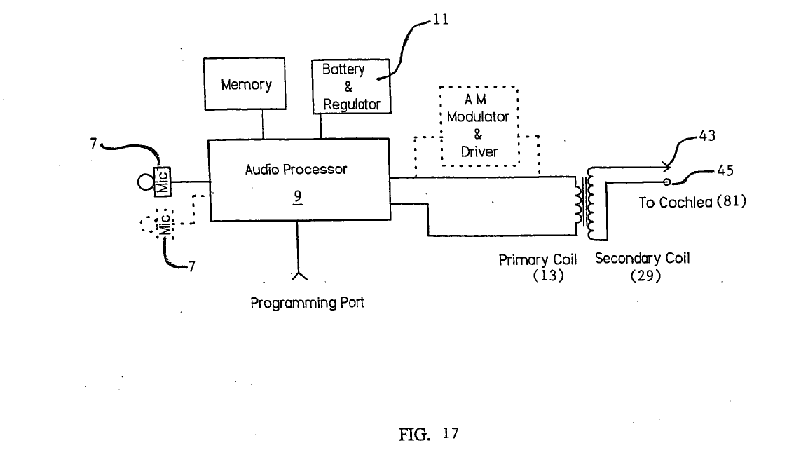

As shown in the figures, and principally FIG. 16, the third component of the cochlear implant is the electrode array 41. The electrode array 41 includes a biocompatible connector 33 for mating to the corresponding connector formed on the interior ear module's housing 27. The electrode may take various forms, such as including a large number of active electrodes for stimulation throughout the entire cochlea. However, it is preferred that the electrode array is a simple structure including a single active electrode 43 and a return electrode 45. Preferably, each of the electrode wires are sheathed in a biocompatible cover. Moreover, it is preferred that the return electrode includes a small conductive mesh region 51 to enable a low impedance tissue connection.

To implant the electrode requires a minor surgical procedure within a doctor's office conducted using a local anesthesia. An incision is made through the tympanic membrane and the electrode array 41 is manually forced through the incision. Thereafter, it is preferred that the active electrode 43 is positioned to engage the cochlea's nerve cells. At the same time, the interior ear module is positioned within the ear canal 73 exterior to the tympanic membrane 77. Over the next days and weeks, the tympanic membrane heals around the electrode array 41 thereby providing a substantially gaseous seal. Upon manual implantation of the exterior ear module, employing ear canal, in-the-ear (concha bowl) or behind-the-ear constructions, the entire cochlear implant is provided for allowing the totally deaf to hear sound.

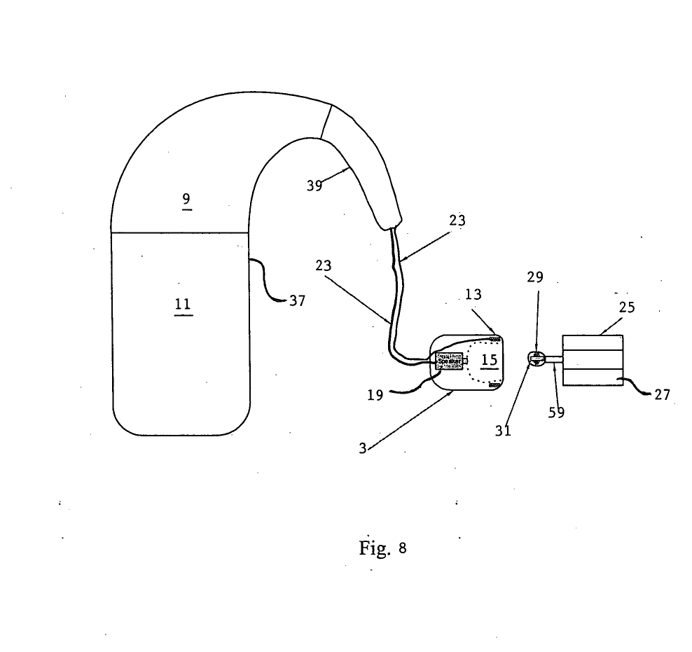

As shown in the figures, numerous modifications can be made to the cochlear ear implant of the present invention. For example, recently it has been understood that low frequency acoustic energy to the tympanic membrane can assist those with hearing in the lower portion of the audio spectrum by providing both electrical and acoustic stimuli. More particularly, it has recently been determined that a significant percentage of cochlear implant candidates retain usable residual hearing in the lower frequency ranges of the audio spectrum. By providing both electrical and acoustic stimuli, significant gains can be obtained by the patient. For example, as shown in FIGS. 8-10, the exterior ear module 3 or behind-the-ear module 37 is constructed to include a speaker assembly 19. Where the cochlear ear implant includes a speaker, preferably the speaker includes audio filters for producing sound only in the lower frequency ranges, while the electrical stimulation through the electrode array is filtered to include only higher frequency stimuli. Preferably, a mutual crossover frequency is established between the acoustic signal spectrum and electrical spectrum. Caution must be exercised so as to avoid acoustic feedback.

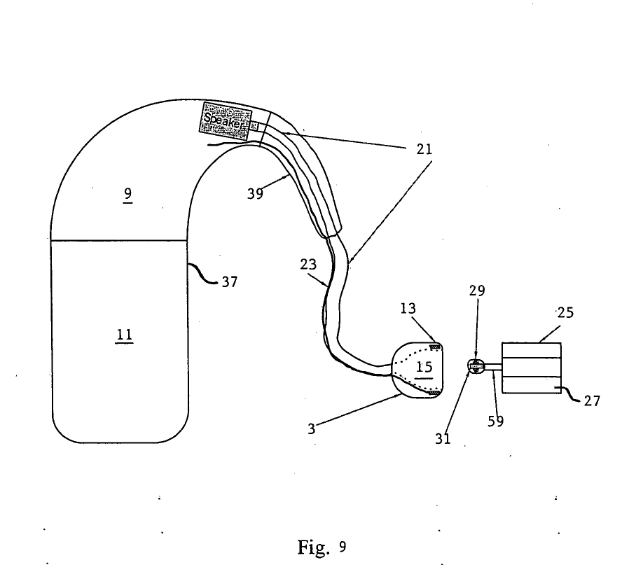

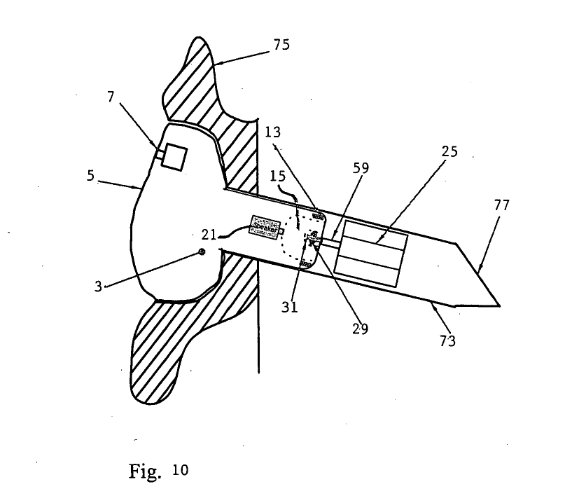

With reference to FIG. 8, in a first embodiment using an acoustic signal, the cochlear ear implant includes a behind-the-ear module 37 and an exterior ear module 3. A speaker is provided within the exterior ear module which produces an acoustic response within the cavity 15. Power is supplied from the power supply 11 to the speaker using a wire connection 23. In an alternative construction shown in FIG. 9, the speaker is positioned within the behind-the-ear module 37 which provides additional space not provided within the exterior ear module 3. In addition, the cochlear ear implant includes acoustic tubing 21 for transmitting pressure waves from the speaker 21 to the exterior ear module's interior cavity 15. Finally, still an additional cochlear implant including acoustic response is illustrated in FIG. 10. This figure illustrates a cochlear ear implant including an exterior ear module sized and positioned to reside within the ear's concha bowl. The exterior ear module is provided with a speaker 21 which produces pressure waves within the cavity 15. Notably, modification to the exterior ear module 3 should be made where the cochlear ear implant produces acoustic signals. For example, instead of including aeration vents, the exterior ear module is preferably sealed, except for a very small opening to provide for pressure relief. The seal prevents acoustic feedbacks from occurring by eliminating the speaker from the microphone's acoustic path. Moreover, substantially closing the ear canal increases the pressure responses to the tympanic membrane produced by the speaker 21.

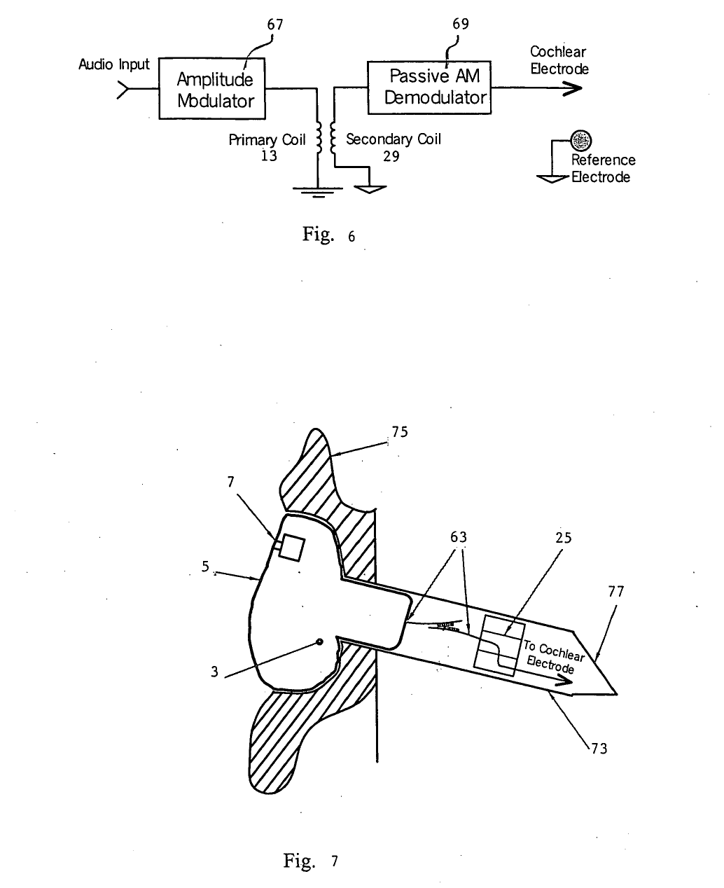

Still additional modifications of the cochlear ear implant can be made. For example, the invention has been described predominantly using digital signal process to produce pulsatile waveforms that are modulated with audio information to power the primary coil of the induction coupling system. However, the cochlear ear implant of the present invention is also capable of using amplitude modulation methods. For example, as shown in FIGS. 6 and 17, the audio input produced by the microphone can be modulated by an amplitude modulator 67 to produce an amplitude modulated electromagnetic field from the primary coil 13. Meanwhile, the interior ear module includes a passive amplitude demodulator 69 for converting the electromagnetic waves into stimulus signals recognizable by the brain through the cochlear nerves.

Still additional modifications of the cochlear implant of the present invention can be made without departing from the spirit and scope of the invention. Having described my invention in such terms to enable those skilled in the art to make and use it, and having identified the presently preferred embodiments thereof,

1. A transcanal cochlear implant for an ear having an auditory canal, a middle ear, a cochlea, a tympanic membrane, and a scala tympanic, the transcanal cochlear implant comprising:
a microphone positioned exterior to the tympanic membrane for converting sound waves into microphone electrical signals;
a processor positioned exterior to the tympanic membrane for converting said microphone electrical signals to stimulus signals adapted to stimulate nerves within the cochlea;
a power source for said microphone and processor; and
an implantable electrode array extending through the tympanic membrane to said cochlea for transmitting said stimulus signals from said processor to stimulate nerves within the cochlea.

2. The transcanal cochlear implant of claim 1 wherein said microphone, processor and power source are disengageable from said electrode array and removable from a patient without surgery.

3. The transcanal cochlear implant of claim 2 wherein said microphone, processor and power source are positioned within the auditory canal.

4. The transcanal cochlear implant of claim 2 wherein said microphone, processor and power source are positioned within an over-the-ear module which is constructed to rest on the ear's auricle.

5. The transcanal cochlear implant of claim 1 further comprising:
a removable exterior ear module including said microphone, processor and power source, said exterior ear module positioned within the exterior portion of said auditory canal and removable from said auditory canal without surgery; and
a semi-permanent interior ear module connectable and disconnectable to said exterior ear module, said interior ear module connecting said exterior ear module to said electrode array for relaying stimulus signals from said processor to said electrode array.

6. The transcanal cochlear implant of claim 5 wherein:
said exterior ear module includes a primary coil for converting signals produced by said processor into electromagnetic signals; and
said interior ear module includes a secondary coil for converting said electromagnetic signals into stimulus signals.

7. The transcanal cochlear implant of claim 1 further comprising a speaker assembly producing acoustic signals.

8. The transcanal cochlear implant of claim 5 further comprising a speaker assembly producing acoustic signals which is located in said exterior ear module.

9. A transcanal cochlear implant for an ear having an auditory canal, a middle ear, a cochlea, a tympanic membrane, and a scala tympanic, the transcanal cochlear implant comprising:
a microphone positioned exterior to the tympanic membrane for converting sound waves into microphone electrical signals;
a processor positioned exterior to the tympanic membrane for converting said microphone electrical signals to stimulus signals adapted to stimulate nerves within the cochlea;
an electrode array extending through the tympanic membrane to said cochlea for transmitting said stimulus signals to stimulate nerves within the cochlea;
a removable exterior ear module including said microphone, processor and power source, said exterior ear module removable from the ear without surgery; and
a semi permanent interior ear module connectable and disconnectable to said exterior ear module, said interior ear module positioned in the auditory canal exterior to the tympanic membrane and connecting said exterior ear module to said electrode array for relaying stimulus signals from said processor to said electrode array.

10. The transcanal cochlear implant of claim 9 wherein:
said exterior ear module is constructed to reside within the ear's auditory canal.

11. The transcanal cochlear implant of claim 9 wherein:
said exterior ear module is constructed to reside within the ear's concha bowl.

12. The transcanal cochlear implant of claim 11 wherein:
said exterior ear module includes a primary coil for converting signals produced by said processor into electromagnetic signals; and
said interior ear module includes a secondary coil for converting said electromagnetic signals into stimulus signals.

13. The transcanal cochlear implant of claim 11 further comprising a speaker assembly producing acoustic signals.

14. The transcanal cochlear implant of claim 12 further comprising a speaker assembly producing acoustic signals which is located in said exterior ear module.

15. A transcanal cochlear implant for an ear having an auditory canal, a middle ear, a cochlea, a tympanic membrane, and a scala tympanic, the transcanal cochlear implant comprising:
a microphone positioned exterior to the tympanic membrane for converting sound waves into microphone electrical signals;
a processor positioned exterior to the tympanic membrane for converting said microphone electrical signals to stimulus signals adapted to stimulate nerves within the cochlea;
an electrode array transmitting signals from said processor through the tympanic membrane to said cochlea for transmitting said stimulus signals to stimulate nerves within the cochlea;
an over-the-ear module including said microphone, processor and power source; and
an interior ear module connectable and disconnectable to said processor, said interior ear module positioned in the auditory canal exterior to the tympanic membrane and connecting said over-the-ear module to said electrode array for relaying stimulus signals from said processor to said electrode array.

16. The transcanal cochlear implant of claim 15 further comprising:
a removable exterior ear module positioned within the exterior portion of said auditory canal and removable from said auditory canal without surgery; and
an interior ear module connectable and disconnectable to said exterior ear module, said interior ear module connecting said exterior ear module to said electrode array for relaying stimulus signals from said processor to said electrode array.

17. The transcanal cochlear implant of claim 16 wherein:
said exterior ear module includes a primary coil for converting signals produced by said processor into electromagnetic signals; and
said interior ear module includes a secondary coil for converting said electromagnetic signals into stimulus signals.

18. The transcanal cochlear implant of claim 15 further comprising a speaker assembly producing acoustic signals.

19. The transcanal cochlear implant of claim 16 further comprising a speaker assembly producing acoustic signals located in said exterior ear module.

20. A method of implanting a cochlear implant in a patient comprising the steps of:
providing a cochlear implant including a microphone positioned exterior to the tympanic membrane for converting sound waves into microphone electrical signals, a processor positioned exterior to the tympanic membrane for converting said microphone electrical signals to stimulus signals adapted to stimulate nerves within the cochlea, a power source for said microphone and processor; and an implantable electrode array extending through the tympanic membrane to said cochlea for transmitting said stimulus signals from said processor to stimulate nerves within the cochlea;
cutting an incision in a patient's tympanic membrane; and
directing the electrode array through the tympanic membrane so that the electrode array engages the patient's cochlea.

21. The method of implanting a cochlear implant in a patient of claim 20 further comprising the step of positioning the microphone, processor and power supply in an auditory canal module within the patient's auditory canal.

22. The method of implanting a cochlear implant in a patient of claim 20 further comprising the step of positioning the microphone, processor and power supply in an in-the-ear module within the patient's ear's concha bowl.

23. The method of implanting a cochlear implant in a patient of claim 20 further comprising the step of positioning the microphone, processor and power supply in a behind-the-ear module behind the patient's pinna.
